(19) United States
(12) Patent Application Publication
(10) Pub. No.: US 2023/0377739 A1
(43) Pub. Date: Nov. 23, 2023

(54) Data Distribution Component of Digital Healthcare Platform

(71) Applicant: Health2047, Inc., Menlo Park, CA (US)

(72) Inventors: Charles Edward George Keith Aunger, Hayward, CA (US); Judy L. Barkal, Menlo Park, CA (US); Gloria N. Cao, San Jose, CA (US); Edison Ting, Celina, TX (US)

(21) Appl. No.: 18/198,686

(22) Filed: May 17, 2023

Related U.S. Application Data
(60) Provisional application No. 63/343,069, filed on May 17, 2022.
(60) Provisional application No. 63/343,071, filed on May 17, 2022.
(60) Provisional application No. 63/343,072, filed on May 17, 2022.
(60) Provisional application No. 63/343,073, filed on May 17, 2022.

(51) Int. Cl. G16H 40/63; G16H 15/00; G06Q 30/018

(52) U.S. Cl. CPC G16H 40/63; G16H 15/00; G06Q 30/018

(57) ABSTRACT

A novel data distribution gateway component in a digital healthcare framework stores physiological sensor data enhanced with contextual data, where such contextual data comprises a physiological sensor data point generated at a device component, a user ID that was added by the device component, a device ID that was added by the device component, a timestamp that was added by device component, medical/code annotation data added by a coordination gateway, clinical validation annotation data added by the coordination gateway, and regulatory compliance annotation data added by the coordination gateway.

### CROSS-REFERENCE TO RELATED APPLICATIONS

The present application claims the benefit of U.S. Provisional Application No. 63/343,069 filed on May 17, 2022; the entire disclosure contents are fully incorporated by reference into the present application. The present application claims the benefit of U.S. Provisional Application No. 63/343,071 filed on May 17, 2022; the entire disclosure contents are fully incorporated by reference into the present application. The present application claims the benefit of U.S. Provisional Application No. 63/343,072 filed on May 17, 2022; the entire disclosure contents are fully incorporated by reference into the present application. The present application claims the benefit of U.S. Provisional Application No. 63/343,073 filed on May 17, 2022; the entire disclosure contents are fully incorporated by reference into the present application.

Applicant of the present application owns U.S. application Ser. No. 17/446,210 filed on Aug. 27, 2021, entitled “Enhanced Authentication Techniques Using Virtual Persona,” which is herein fully incorporated by reference in its entirety.

### BACKGROUND OF THE INVENTION

### Field of Invention

The present invention generally relates to the field of healthcare platforms. More specifically, the present invention is related to a novel data distribution gateway component of a digital healthcare platform.

### Discussion of Related Art

FIG. 1 depicts the current state-of-the-art in remote patient care. Element 102 represents a medical device (e.g., a blood pressure monitor, a glucometer, or other medical devices) that is configured to collect physiological data associated with a patient. In current state-of-the-art systems, physiological data is measured (via one or more sensors on the medical device) at the client-side (i.e., the patient side) and is transmitted, typically over the Internet, via an encrypted channel 104 to a data platform at a remote location. The data platform logs the received data in a logs database 106.

In current state-of-the-art systems, data that gets sent from the client-side typically comprises a unique device identifier (ID) in addition to the physiological data. For example, in the case of a blood pressure monitor, the client-side device transmits systolic/diastolic pressures, along with additional information such as pulse rates, etc. When such information is received by the data platform, it logs this information into the logs database 106. In this setup, an additional correlation or mapping is needed to associate the data received and stored with the device ID identifying the specific device that transmitted the data and a user identifier (ID) identifying which user is associated with the transmitted data. Data identifying which device transmitted the data is stored in a Device IDs database 108 and data identifying which user is associated with the transmitted data is stored in a User IDs database 110.

As one example, a first table may be maintained that maps a second table of device IDs and a third table of user IDs, where the data platform performs a look-up of a particular user ID to determine a corresponding device ID (device IDs if there are a plurality of devices associated with a particular user) or may perform a look-up of the device ID to determine the associated user ID. Accordingly, when the clinician accesses the data platform (via, for example, an application such as a patient dashboard) to review physiological data associated with a given patient, the data platform (at the back-end) correlates the user ID (obtained from the User IDs database 110) of the given patient with the device ID of the given patient (obtained from the User IDs database 108), retrieves data associated with the given patient based on such correlation, and renders such retrieved data at the clinician's end. In such state-of-the-art systems, a lot of time/resources are spent in resolving such correlations so that the physician views data associated with the appropriate patient that he/she is requesting data for.

Also, in such state-of-the-art systems, much of the correlation/mapping (or steps leading up to the correlation/mapping) is performed after all of the device data is sent over to the data platform. As a result of this, some of the client-side information gets lost. For example, by the time the device data is sent to the data platform, the specific person taking the measurement is lost as that data is not sent to the data platform along with the physiological data collected by the device. This leads such state-of-the-art systems to maintain mapping/correlation data after the physiological data is collected, which may not be desirable.

Another problem with such state-of-the-art systems is that they are unable to distinguish who specifically in a household used the device. Clinicians experience problems when they do not know who was using the device at the time physiological data was collected, as there may have been additional members of the household that may have had access to the same device. A workaround that may be used is to check if measurements deviate from historical data, but such workarounds are inherently not accurate. Likewise, having clinicians look for such deviations also disrupts their actual workflow as they are no longer focused on analyzing patient data, but are rather focused on whether or not the patient data corresponds to the right person in the household (especially in instances where collected physiological data shows a large deviation).

Further, in the state-of-the-art set up, the services that the clinician provides are billable. Accordingly, the time the clinician spends reviewing the data and/or talking with the patient is billable through a central location such as the Centers for Medicare and Medicaid Services (CMS).

There is, thus, a billing component where the data (that is collected in the data platform) and transactions associated with a patient (in terms of the time the physician is spending with the patient and the time the physician is spending reviewing the data from the devices associated with that patient) are used in processing claims, usually by a claims department.

Such claims departments use one or more pre-determined codes (e.g., Current Procedural Terminology (CPT®) codes, where each CPT® code is a five-digit numeric code assigned to each task and service a healthcare provider offers (e.g., medical, surgical, and diagnostic services)). While examples are provided with CPT codes, it should be noted that other pre-determined codes may be used without departing from the scope of the present invention. Insurers use the numbers to determine how much money to pay a physician (e.g., certain CPT® codes allow a physician to bill 20 or 40 minutes of his/her time). Such CPT® codes may be stored in a CPT® codes database 112. During claims processing, a determination needs to be made regarding what data may be mapped to which CPT® code, and from that determine what the billable details are.

Another important code is the International Classification of Diseases (ICD©) code. The World Health Organization (WHO) defines ICD-11© as a classification and terminology system that “allows the systematic recording, analysis, interpretation and comparison of mortality and morbidity data collected in different countries or regions and at different times” and “ensures semantic interoperability and reusability of recorded data for the different use cases beyond mere health statistics, including decision support, resource allocation, reimbursement, guidelines and more.” Broadly, ICD-11© codes refer to the condition being treated (i.e., high blood pressure, diabetes, etc.). ICD-11© codes ensure that a patient gets proper treatment and is charged correctly for any medical services they receive. ICD-11© codes are used in billing (where billing is based on rules stored in a billing rules databases 116), treatments, and statistics collection. Having the right code is important to ensure that standardized treatment for a medical issue is delivered and that medical expenses are reimbursed. Such ICD-11© codes may be stored in an ICD-11© codes database 114. When a healthcare provider submits a bill to an insurance company for reimbursement, each service is described by the CPT® code, and this CPT® code is matched to an ICD-11© code. If the two codes don't align correctly with each other, the insurance company may deny payment.

One problem with CPT® codes is that a plurality of CPT® codes may apply to the same data set. As an example, a first patient may send data in digital form, while the same patient may send another set of data by manually entering it. It happens that those two mediums (i.e., digital vs manual) apply to two different CPT® codes, and they have different billing requirements. While the clinician just wants to see the data and not necessarily the billing codes and the manner in which the data was sent, often times the claims department has to consult back with the clinician to see if this is digitally or manually collected. In many instances, the software development has to go in and determine how it was collected.

Another problem with standardized codes, such as CPT® codes, (while not knowing the codes beforehand at the edge or client-facing application and having to find the mapping later at the backend/data platform side) is that rules that apply for CPT® codes tend to change from time to time. For example, let's take code 99454 (a billing code for supplying and monitoring patients with remote patient monitoring devices) as an example. A couple of years ago, the rule was that each patient should at least submit two data points in a month at a minimum (as long as someone reviewed the data), which allows the physician providing the service to that patient to bill for that CPT® code. However, it was recently revised to state that the same CPT® code now requires the patient to submit 16 data points in a month. State-of-the-art systems lack sophistication in determining what rules apply to data collected prior to a rule change and what rules apply for data collected since the rule has gone into effect.

Such state-of-the-art healthcare frameworks also do not provide an easy manner in which to incorporate new data sources (i.e., physiological data from non-clinically validated devices) without adding more complexity. Yet data sources from these devices could provide supporting evidence that may be useful for the healthcare team.

Such problems with state-of-the-art systems cause a lot of inefficiencies and problems in terms of the way information is processed, and such inefficiencies and problems lead to various errors.

Embodiments of the present invention are an improvement over prior art systems and methods.

### SUMMARY OF THE INVENTION

In one embodiment, the present invention provides a data distribution gateway of a digital healthcare framework comprising: (a) a receiver configured to receive annotated physiological sensor data from a coordination gateway in the digital healthcare framework; (b) an event logs storage storing the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and (c) one or more application programming interfaces (APIs) providing one or more users access to the event logs storage based on stored user permissions associated with each of the one or more users.

In another embodiment, the present invention provides a method as implemented in a data distribution gateway of a digital healthcare framework, the method comprising: (a) receiving annotated physiological sensor data from a coordination gateway in the digital healthcare framework; (b) storing, in an event logs storage, the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and (c) providing one or more application programming interfaces (APIs) configured to allow one or more users access to the event logs storage based on stored user permissions associated with each of the one or more users.

In yet another embodiment, the present invention provides an article of manufacture comprising non-transitory computer storage medium storing computer readable program code which, when executed by a computer, implements a method as implemented in a data distribution gateway of a digital healthcare framework, the medium comprising: (a) computer readable program code receiving annotated physiological sensor data from a coordination gateway in the digital healthcare framework; (b) computer readable program code storing, in an event logs storage, the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and (c) computer readable program code providing one or more application programming interfaces (APIs) configured to allow one or more users access to the event logs storage based on stored user permissions associated with each of the one or more users.

### BRIEF DESCRIPTION OF THE DRAWINGS

The present disclosure, in accordance with one or more various examples, is described in detail with reference to the following figures. The drawings are provided for purposes of illustration only and merely depict examples of the disclosure. These drawings are provided to facilitate the reader's understanding of the disclosure and should not be considered limiting of the breadth, scope, or applicability of the disclosure. It should be noted that for clarity and ease of illustration these drawings are not necessarily made to scale.

FIG. 1 depicts a prior art remote patient monitoring platform.

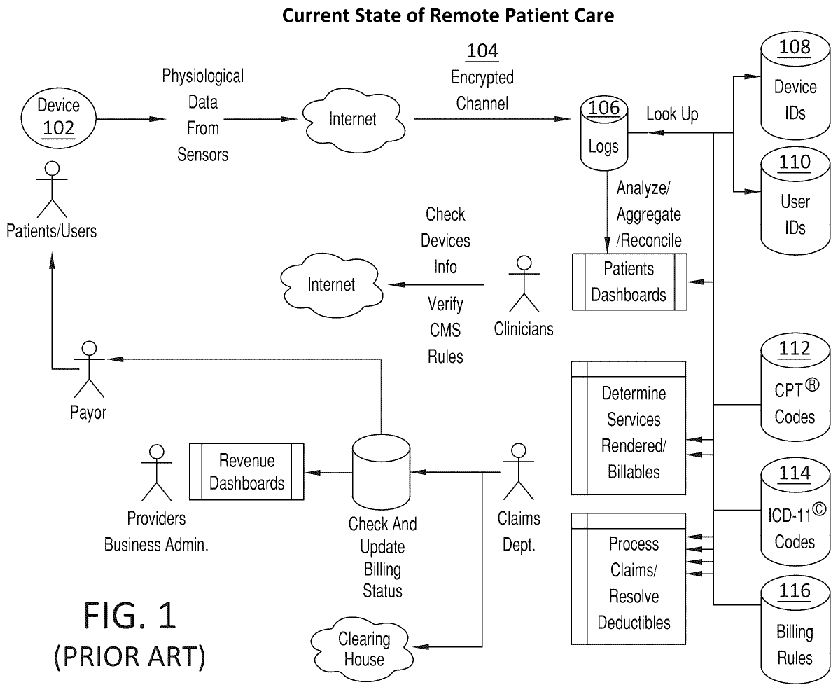

FIG. 2 depicts the present invention's digital healthcare framework application.

### DESCRIPTION OF THE PREFERRED EMBODIMENTS

While this invention is illustrated and described in a preferred embodiment, the invention may be produced in many different configurations. There is depicted in the drawings, and will herein be described in detail, a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the present invention.

Note that in this description, references to “one embodiment” or “an embodiment” mean that the feature being referred to is included in at least one embodiment of the invention. Further, separate references to “one embodiment” in this description do not necessarily refer to the same embodiment; however, neither are such embodiments mutually exclusive, unless so stated and except as will be readily apparent to those of ordinary skill in the art. Thus, the present invention can include any variety of combinations and/or integrations of the embodiments described herein.

The present invention provides a digital healthcare framework that ensures trust and integrity of the data, and enables true ownership of data, defining which patient the data corresponds to and what are the permitted uses for that data. In the present invention's digital healthcare framework, substantial work is performed at the edge (i.e., the client or patient side) to determine: (1) what data is collected by the sensors and what is the context of the data collected, (2) who was using the device, and (3) what rules apply to the collected data. The present invention's digital healthcare framework, at the highest level, provides for streaming data collected at the edge (i.e., the client or patient side) in a more intelligent way by attaching a context to that data, as will be explained below.

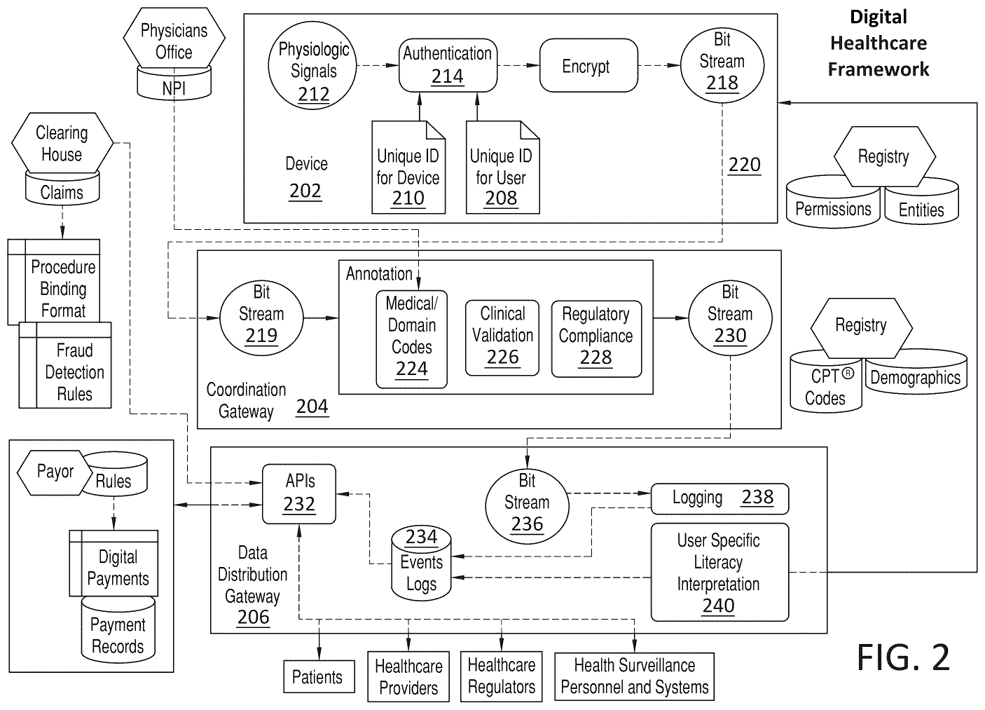

FIG. 2 depicts an overview of the present invention's digital healthcare framework 200 comprising the following components: (1) device component 202; (2) coordination gateway component 204, and (3) data distribution gateway component 206. The present patent application focuses on the novelty aspects of the data distribution gateway component 204.

The concurrently filed non-provisional application titled “Device Component of Digital Healthcare Platform”, with Serial No. XX/XXX,XXX, which is fully incorporated by reference in its entirety, provides more details on device component 202, and the concurrently filed non-provisional application titled “Coordinating Gateway Component of Digital Healthcare Platform”, with Serial No. XX/XXX,XXX, which is fully incorporated by reference in its entirety, provides more details on the coordination gateway 204.

Device 202 represents a medical device (e.g., a blood pressure monitor, a glucometer, oximeter, spirometer, oxygen nebulizer, stethoscope, electrocardiogram (ECG) device, thermometer, activity tracker, weighing scale, or other medical devices) that is configured to collect physiological data associated with a patient. It should be noted that while specific examples of devices such as blood pressure monitors, glucometers, etc. are used throughout the specification, these are non-limiting examples of such devices. Any device that is capable of collecting and transmitting (to either a remote location by itself or via a proxy device) one or more physiological data points is envisioned for use with the present invention.

Unlike the state-of-the-art systems, in the digital healthcare framework of FIG. 2, a unique user identifier (ID) 208 based on the identity of the user of the device 202 and a unique device identifier (ID) 210 that uniquely identifies the device are assigned as context to the physiological data collected at the device 202.

Physiological sensor data 212 is collected by device 202, but before the device sends the collected physiological sensor data 212, the device attaches to a data frame (having data associated with the collected physiological sensor data 212), information about “who” (i.e., which user was using device 200 when the collected physiological sensor data 212 was collected) and “what” (i.e., what device is collecting the physiological sensor data 212).

The authentication engine 214 addresses the “who” aspect above by authenticating the user prior to data collection/data transmission, where such features are implemented and performed at the device level. Once the user is identified by the authentication engine 214, a unique user ID 208 is identified for attaching to a data frame.

Physiological data is measured (via one or more sensors on device 202) at the client-side (i.e., the patient side), and context information such as unique device ID 210 and unique user ID 208 are added to such physiological data, where collected data is enhanced with context information. Such enhanced data is then encrypted, via encryption engine 216, to form bitstream 218 which is typically transmitted over the Internet, via an encrypted channel 220 to the present invention's coordination gateway 204 at a remote location.

Coordination gateway 204 receives the encrypted bitstream received from the device component 202 and annotates the received bitstream with additional information which will be described below. The present invention's coordination gateway 204 comprises an annotation component 222 that further comprises a medical/domain codes annotator component 224, clinical validation annotator component 226, and regulatory compliance annotator component 228.

The medical/domain codes annotation component 224 identifies one or more pre-determined codes that may be associated with the received data and tags the data with such identified pre-determined codes.

The clinical validation annotator component 226 identifies information regarding whether or not the device (that the data corresponds to) is a clinically validated device and the clinical validation annotator component 226 tags the received data with such identified information. This component lets the backend (i.e., data distribution component 206) know whether or not the device from which the data is originating is a clinically validated device. However, it should be noted that the novel digital healthcare framework does not necessarily preclude data from a device that is not clinically validated but, rather, makes data from such devices available with annotations that may be extracted at the backend to let clinicians know that such data is from a device that is not clinically validated. For example, sensor data transmitted from a smartwatch device (which falls under the category of a device that is not clinically validated) may still provide additional context for the patient and, accordingly, the present invention leaves the decision of whether or not to use that data to the clinician. However, without tagging such validation information (as to whether data is from a clinically validated device or not), the backend would not know the availability of such information to present to a clinician.

Clinical validation annotator component 226 may communicate with an external website that contains a list of clinically validated devices to see if the device from which the data was received is in such a list. Clinical validation annotator component 226 may store such information locally within the coordination gateway 204.

The regulatory compliance annotator component 228 verifies if the device from which the data originated is compliant with regulatory rules. For example, the device from which the data originated may be a device that is under recall. Such information may be identified by the regulatory compliance annotator component 228 and any such identified information may be added to the received data.

It should be noted that the dynamic nature of the coordination gateway component 204 allows for a real-time check on a device's clinical validation status or regulatory compliance status as such a real-time check is just a service call to another website or remote server. Even in instances where the recall information of a given device has not permeated healthcare systems, the present invention's coordination gateway may do such a check in real-time and annotate device data with such information (annotating, for example, that it's been recalled by regulatory authorities). In such instances where the device manufacturers are very slow in being able to update software (not just because of their own slowness, but sometimes health systems prevent them from updating information), the present invention's coordination gateway provides a way to annotate device data with the goal to inform the physician whether or not the device (where the data originated from) has been recalled, who may then proactively reach out to the patient who may or may not be aware of such a recall.

The output of the annotation component 222 (when the received data has been annotated with additional information about medical domain codes, clinical validation information, and regulatory compliance information) is transmitted to a data distribution gateway component.

The present invention's data distribution gateway 206 comprises one or more APIs 232, logging component 238, and an event logs storage 234 (a nonlimiting example of this could be a database). Logging component 238 receives bitstream 236 containing the annotated data (which is self-describing as it has annotations that describe it) from the coordination gateway and logs the annotated data in the event logs storage 234. APIs 232 allow for appropriate external parties to access the event logs storage 234. APIs 232 respond to requests regarding events. One type of request can be a yes/no response that an event exists (with optional parameters that describe who, when, and duration of the event). Another type of response could be the number of events that match a given criteria (e.g., how many blood pressure measurements were normal today?). User permissions (from the Registry) determine whether such a query of events is permitted and what data the API can return.

Event logs storage 234, therefore, receives self-describing data where all the work to describe the data (i.e., by both the device component 202 that adds among other things, the unique device ID and unique user ID, and the coordination gateway component 204 that adds among other things, the medical/domain code annotation, the clinical validation annotation, and the regulatory compliance annotation) is done prior to its arrival at the data distribution gateway 206 in real-time and logs such real-time data in an efficient manner in the event logs storage 234.

Each data point stored in the event logs storage 234 could have, for example, the following details: a physiological sensor data point generated at the device component 202 (e.g., a blood pressure reading generated at the device component 202), a user ID that was added by the device component 202 (e.g., user name for which the blood pressure reading applies), a device ID that was added by the device component 202, a timestamp that was added by device component 202, medical/code annotation data added by the medical/domain codes annotator component 224 of the annotation component 222 of the coordination gateway 204, clinical validation annotation data added by the clinical validation annotator component 226 of the annotation component 222 of the coordination gateway 204, and regulatory compliance annotation data added by the regulatory compliance annotator component 228 of the annotation component 222 of the coordination gateway 204. In one non-limiting example, the present invention envisions a patient-centric knowledge graph where events are created/added to nodes on the graph. It is important that data associated with a device can persist even if the device gets reassigned to a different patient. Separate data constructs can be developed to improve performance for a “common library” or prioritized set of event queries.

The user-specific library interpretation component 240 of the data distribution gateway 206 provides authorized data (i.e., authorized for view by the particular user) stored in the event logs storage to a user via a custom interface that is targeted toward that particular user. For example, if the user is a patient, a set of custom interfaces may be provided by the user-specific literacy interpretation component 240 of the data distribution gateway 206, and if the user is a physician, another set of custom interfaces may be provided by the user-specific literacy interpretation component 240 of the data distribution gateway 206. In the instance where the user is a layman user, the user-specific literacy interpretation component 240 knows this is a patient (and they are not a healthcare professional), so they need layman's terminology. Accordingly, the user-specific literacy interpretation component 240 may provide interfaces that provide a simple explanation of benefits to explain what a procedure was that the patient underwent, where such interfaces may even suggest any actions that they might want to take based on what they are viewing. For example, a user who wants to view their blood-pressure data may want to know if their value is too high or too low. The literacy interpretation component 240 provides such additional context to the data being viewed depending on the type of user.

Interfaces rendered by the data distribution gateway may differ, for example, depending on a given user's healthcare skills and training or a given user's audio, visual, and language capabilities.

Such interfaces may also provide action items for the user as well. One example of such an action might be to contact his/her physician. Another example of such an action may be a behavioral recommendation (e.g., reduce salt intake or increase activity/steps).

Additional interfaces that may be displayed via device component 202 may also be found in the concurrently filed provisional patent application titled “Graphical User Interfaces (GUIs) Associated with a Data Distribution Gateway of a Digital Healthcare Platform”, with Serial No. XX/XXX,XXX, which is fully incorporated by reference.

Another key aspect of the data distribution gateway is that permissions for use of data stored in the event logs storage 234 can be dynamically assigned or revoked. For example, should a physician leave his current job at a first healthcare facility to join a second healthcare facility, that physician should not be able to view data associated with a patient he/she was assigned to at the first healthcare facility (where permissions would be revoked), while the same physician should be able to view data associated with a patient, he/she was assigned to at the second healthcare facility (where permissions need to be assigned). The data distribution gateway component 206 gets such permissions from the Registry on a per-user request. Accordingly, the Registry keeps permissions up-to-date for registered users.

CMS defines a national provider identifier (NPI) as a Health Insurance Portability and Accountability Act (HIPAA) administrative simplification standard that is a unique identification number for covered healthcare providers. Covered healthcare providers and all health plans and healthcare clearinghouses must use the NPIs in the administrative and financial transactions adopted under HIPAA. The NPI is a 10-position, intelligence-free numeric identifier (10-digit number). This means that the numbers do not carry other information about healthcare providers, such as the state in which they live or their medical specialty. The NPI must be used in lieu of legacy provider identifiers in the HIPAA standards transactions. In FIG. 2, the physician's office's NPI may be used by the medical/domain codes annotation component to tag data received from device component 202. NPI's are associated with providers in the Registry Component of Digital Healthcare Framework.

The clearinghouse (handling claims) along with the fraud detection (rules) component collectively forms a rules engine that is used to check event logs (stored in the event logs storage 234) to detect fraud (based on stored detection rules). The procedure (binding format) component allows, for example, the binding of two pre-determined codes that may be used together in claims handling in the digital healthcare framework.

The novel digital healthcare framework also provides for a digital payment system. When a bill is ready for the payor to pay, the stored rules define forms of acceptable digital payment methods that the payor may use. Upon successful payment via one of the acceptable digital payment methods, payment records are stored in a payments records database.

A myriad of users may access data distribution gateway component 206 where such users include, but are not limited to, patients, providers, clearinghouses, payors, digital payment systems, healthcare regulators, health surveillance personnel and systems (e.g., real-world data (RWD)/real-world evidence (RWE)), etc.

In one embodiment, the present invention provides a data distribution gateway of a digital healthcare framework comprising: (a) a receiver configured to receive annotated physiological sensor data from a coordination gateway in the digital healthcare framework; (b) an event logs storage storing the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and (c) one or more application programming interfaces (APIs) providing one or more users access to the event logs storage based on stored user permissions associated with each of the one or more users.

In another embodiment, the present invention provides a method as implemented in a data distribution gateway of a digital healthcare framework, the method comprising: (a) receiving annotated physiological sensor data from a coordination gateway in the digital healthcare framework; (b) storing, in an event logs storage, the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and (c) providing one or more application programming interfaces (APIs) configured to allow one or more users access to the event logs storage based on stored user permissions associated with each of the one or more users.

In yet another embodiment, the present invention provides an article of manufacture comprising non-transitory computer storage medium storing computer readable program code which, when executed by a computer, implements a method as implemented in a data distribution gateway of a digital healthcare framework, the medium comprising: (a) computer readable program code receiving annotated physiological sensor data from a coordination gateway in the digital healthcare framework; (b) computer readable program code storing, in an event logs storage, the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and (c) computer readable program code providing one or more application programming interfaces (APIs) configured to allow one or more users access to the event logs storage based on stored user permissions associated with each of the one or more users.

Non-limiting examples of such codes may be Current Procedural Terminology or CPT codes; however, it should be noted that other pre-determined codes may be used without departing from the scope of the present invention.

The above-described features and applications can be implemented as software processes that are specified as a set of instructions recorded on a computer readable storage medium (also referred to as computer readable medium). When these instructions are executed by one or more processing unit(s) (e.g., one or more processors, cores of processors, or other processing units), they cause the processing unit(s) to perform the to actions indicated in the instructions. Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor. By way of example, and not limitation, such non-transitory computer-readable media can include flash memory, RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. The computer readable media does not include carrier waves and electronic signals passing wirelessly or over wired connections.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few.

In this specification, the term “software” is meant to include firmware residing in read-only memory or applications stored in magnetic storage or flash storage, for example, to a solid-state drive, which can be read into memory for processing by a processor. Also, in some implementations, multiple software technologies can be implemented as sub-parts of a larger program while remaining distinct software technologies. In some implementations, multiple software technologies can also be implemented as separate programs. Finally, any combination of separate programs that together implement a software technology described here is within the scope of the subject technology. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

These functions described above can be implemented in digital electronic circuitry, in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, for example microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic or solid state hard drives, read-only and recordable BluRay® discs, ultra density optical discs, any other optical or magnetic media, and floppy disks. The computer-readable media can store a computer program that is executable by at least one processing unit and includes sets of instructions for performing various operations. Examples of computer programs or computer code include machine code, for example is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, for example application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

It is understood that any specific order or hierarchy of steps in the processes disclosed is an illustration of example approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components illustrated above should not be understood as requiring such separation, and it should be understood that the described program components and systems can generally be integrated together in a single software product or packaged into multiple software products.

Various modifications to these aspects will be readily apparent, and the generic principles defined herein may be applied to other aspects. Thus, the claims are not intended to be limited to the aspects shown herein, but is to be accorded the full scope consistent with the language claims, where reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject technology.

A phrase, for example, an “aspect” does not imply that the aspect is essential to the subject technology or that the aspect applies to all configurations of the subject technology. A disclosure relating to an aspect may apply to all configurations, or one or more configurations. A phrase, for example, an aspect may refer to one or more aspects and vice versa. A phrase, for example, a “configuration” does not imply that such configuration is essential to the subject technology or that such configuration applies to all configurations of the subject technology. A disclosure relating to a configuration may apply to all configurations, or one or more configurations. A phrase, for example, a configuration may refer to one or more configurations and vice versa.

The various embodiments described above are provided by way of illustration only and should not be construed to limit the scope of the disclosure. Those skilled in the art will readily recognize various modifications and changes that may be made to the principles described herein without following the example embodiments and applications illustrated and described herein, and without departing from the spirit and scope of the disclosure.

While this specification contains many specific implementation details, these should not be construed as limitations on the scope of any invention or of what may be to claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As noted above, particular embodiments of the subject matter have been described, but other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

### CONCLUSION

A system and method have been shown in the above embodiments for the effective implementation of a data distribution component of digital healthcare platform. While various preferred embodiments have been shown and described, it will be understood that there is no intent to limit the invention by such disclosure, but rather, it is intended to cover all modifications falling within the spirit and scope of the invention, as defined in the appended claims. For example, the present invention should not be limited by software/program, computing environment, or specific computing hardware.

1. A data distribution gateway of a digital healthcare framework comprising:
(a) a receiver configured to receive annotated physiological sensor data from a coordination gateway in the digital healthcare framework;
(b) an event logs storage storing the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and
(c) one or more application programming interfaces (APIs) providing one or more users access to the event logs storage based on stored user permissions associated with each of the one or more users.

2. The data distribution gateway of claim 1, a user within the one or more users is a patient, wherein the patient determines one or more permissions associated with which other users within the one or more users have access to data associated with the patient that is stored in the event logs storage.

3. The data distribution gateway of claim 1, wherein the data distribution gateway further comprises a user-specific literacy interpretation component providing event logs storage data for a given user in the one or more users based on one or more permissions associated with the given user via a custom interface targeting the given user.

4. The data distribution gateway of claim 3, wherein the given user is a patient, the user-specific literacy interpretation component provides the custom interface targeting the patient.

5. The data distribution gateway of claim 3, wherein the given user is a physician, the user-specific literacy interpretation component provides the custom interface targeting the physician.

6. The data distribution gateway of claim 3, wherein the user-specific literacy interpretation component provides the custom interface depending on a given user's healthcare skills and/or training.

7. The data distribution gateway of claim 3, wherein the user-specific literacy interpretation component provides the custom interface depending on a given user's audio, visual, and/or language capabilities.

8. The data distribution gateway of claim 3, wherein the custom interface provides a recommendation.

9. The data distribution gateway of claim 1, wherein the event logs storage stores data representative of a patient-centric graph where events are created/added to nodes on the patient-centric graph.

10. The data distribution gateway of claim 1, wherein the stored user permissions for a given user in the one or more users are dynamically assigned or revoked when a role associated with the given user changes within the digital healthcare framework.

11. The data distribution gateway of claim 1, wherein the data distribution gateway communicates with a rules engine to identify fraud within data stored in the event logs storage.

12. The data distribution gateway of claim 1, wherein the data distribution gateway communicates with a digital payment system to handle a digital payment by a given user within the one or more users of the digital healthcare framework wherein, upon successful payment, a payment record is stored in a payment record database.

13. The data distribution gateway of claim 1, wherein each user in the one or more users are any of the following: patient, healthcare provider, clearinghouse, payor, digital payment system, healthcare regulator, a registered user within the digital healthcare framework, and health surveillance personnel/system.

14. A method as implemented in a data distribution gateway of a digital healthcare framework, the method comprising:
(a) receiving annotated physiological sensor data from a coordination gateway in the digital healthcare framework;
(b) storing, in an event logs storage, the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and
(c) providing one or more application programming interfaces (APIs) configured to allow one or more users access to the event logs storage based on stored user permissions associated with each of the one or more users.

15. The method of claim 14, wherein the data distribution gateway further comprises a user-specific literacy interpretation component providing event logs storage data for a given user in the one or more users based on one or more permissions associated with the given user via a custom interface targeting the given user.

16. The method of claim 15, wherein when the given user is a patient, the user-specific literacy interpretation component provides the custom interface targeting the patient, and when the given user is a physician, the user-specific literacy interpretation component provides the custom interface targeting the physician.

17. The method of claim 15, wherein the user-specific literacy interpretation component provides the custom interface depending on a given user's healthcare skills and/or training.

18. The method of claim 15, wherein the user-specific literacy interpretation component provides the custom interface depending on a given user's audio, visual, and/or language capabilities.

19. The method of claim 14, wherein the stored user permissions for a given user in the one or more users are dynamically assigned or revoked when a role associated with the given user changes within the digital healthcare framework.

20. An article of manufacture comprising non-transitory computer storage medium storing computer readable program code which, when executed by a computer, implements a method as implemented in a data distribution gateway of a digital healthcare framework, the medium comprising:
(a) computer readable program code receiving annotated physiological sensor data from a coordination gateway in the digital healthcare framework;
(b) computer readable program code storing, in an event logs storage, the received annotated physiological sensor data, the annotated physiological sensor data comprising a unique device identifier (UDI) identifying a medical device component associated with the annotated physiological sensor data, and a unique user identifier (UUI) uniquely identifying a user of the medical device component, the annotated physiological sensor data tagged with any of, or a combination of, the following: (i) one or more pre-determined codes identified by a medical/domain codes annotator component; (ii) clinical validation information identified by a clinical validation annotator component; and (iii) regulatory compliance information identified by a regulatory compliance annotator component, wherein the medical device component, the coordination gateway, the medical/domain codes annotator component, the clinical validation annotator component, and the regulatory compliance annotator component located remotely from the data distribution gateway; and
(c) computer readable program code providing one or more application programming interfaces (APIs) configured to allow one or more users access to the event logs storage based on stored user permissions associated with each of the one or more users.
